(19) United States
(12) Patent Application Publication
(10) Pub. No.: US 2022/0369975 A1
(43) Pub. Date: Nov. 24, 2022

(54) SYSTEM INCLUDING MOBILE DEVICE ACCESSORY FOR DETERMINING STRESS LEVELS AND TREATING THE SAME

(71) Applicant: The Aga Khan University, Karachi (PK)

(72) Inventors: Saleem Sayani, Wynnewood, PA (US); Muhammad Abdul Muqeet, Karachi (PK); Hafiz Imtiaz Ahmed, Karachi (PK); Murad Moosa Khan, Karachi (PK); Sibgha Naseem Malik, Karachi (PK)

(21) Appl. No.: 17/411,669

(22) Filed: Aug. 25, 2021

(30) Foreign Application Priority Data
May 18, 2021 (PK) 360/2021

(51) Int. Cl. A61B 5/16; A61B 5/00; A61B 5/01; A61B 5/024; A61B 5/0533; A61B 5/389; A61B 5/0205

(52) U.S. Cl. CPC A61B 5/165; A61B 5/6898; A61B 5/01; A61B 5/02416; A61B 5/0533; A61B 5/389; A61B 5/0008; A61B 5/02055; A61B 2562/0271

(57) ABSTRACT

Disclosed herein is a system, including a mobile device; a sensor module attached to the mobile device, the sensor module including a temperature sensor, an infrared sensor, at least one conductance sensor, and at least one electromyography sensor; and an application installed on the mobile device, wherein during operation the application causes the mobile device to receive data from one or more of the temperature sensor, the infrared sensor, the at least one conductance sensor, and the at least one electromyography sensor, and to determine information about a stress level of a human subject based on the received data.

### RELATED APPLICATION

This disclosure claims priority to Pakistan Patent Application No. 360/2021, entitled “System Including Mobile Device Accessory for Determining Stress Levels and Treating the Same” and filed on May 18, 2021, the entire contents of which is incorporated herein by reference.

### FIELD

The disclosure relates to a health monitoring system and more specifically, to a health monitoring and tracking system for detecting and monitoring stress levels.

### BACKGROUND

In psychology, stress is a feeling of emotional strain and pressure and is a phenomena that has become a part of daily life for many individuals. When an individual experiences stress, the body releases stress hormones, such as cortisol and adrenaline. The release of these stress hormones result in physiological and psychological symptoms including increased heart rate, blood pressure, breathing rate and elevated feelings of anxiety, and panic. Continued exposure to heightened stress response hormones is an important contributing factor in major health issues. These can include heart disease, high blood pressure, heart attacks, and stroke. Further, chronic stress can cause or exacerbate many serious mental health problems, such as depression, anxiety, and personality disorders. Common stress management practices can include medicinal, physical, or therapeutic treatments.

### SUMMARY

This disclosure features a system for determining and tracking changes in user stress levels. The system includes an accessory for a mobile device that can be incorporated into a cover for the mobile device. The accessory includes sensors to collect heart rate, temperature, and galvanic skin response (GSR). In combination with an app installed on the mobile device, the system determines the stress level of a user based on user data obtained using the accessory. The system obtains the data and saves the data locally or on a networked location, thereby establishing a historical stress level of the individual by analyzing collected data with a stress-determination algorithm. Upon detecting an elevated stress level, the system can provide the user with a visual and/or audio treatment, such as a calming video, and display the treatment on the mobile device's display, thereby activating a relaxation response of a user, e.g., to lower cortisol levels, blood pressure and/or heart rate.

The system can further include electromyography sensors (EMG) to monitor the user's muscle tension, which can also be used to determine and monitor user stress level. Attaching the EMG sensors to one or more muscle groups can allow the system to conduct an EMG-based stress assessment and monitor changes responsive to a relaxation response induced via a visual treatment. The assessment can be performed contemporaneously with the treatment and can terminate the treatment, responsive to decreased muscle tension, when a threshold stress level has been reached. In addition, the system can further conduct a fatigue assessment using the same sensors to monitor changes responsive to tiredness and induce a stimulating treatment, and terminate the treatment in response to received sensor values.

In general, in a first aspect, disclosed herein is a system, including a mobile device; a sensor module attached to the mobile device, the sensor module including a temperature sensor, an infrared sensor, at least one conductance sensor, and at least one electromyography sensor; and an application installed on the mobile device, wherein during operation the application causes the mobile device to receive data from one or more of the temperature sensor, the infrared sensor, the at least one conductance sensor, and the at least one electromyography sensor, and to determine information about a stress level of a human subject based on the received data.

In some embodiments, the sensor module further can include a casing defining an opening for receiving and encasing the mobile device. The sensor module further can include a transceiver housed by the casing. The electromyography sensor being detachably secured to the sensor module. The electromyography sensor can be in wireless communication with the sensor module when detached from the mobile device. The electromyography sensor can be in wired communication with the sensor module.

In some embodiments, the system can further include a visual module for displaying to the human subject a treatment program, can include at least one processor, and a display, wherein the visual module receives a command from the device to play the treatment program on the display. The treatment program can include a visual program, or an audio program.

In some embodiments, the application can be configured to determine information about the stress level of the human subject through operations can include: receiving, from one or more sensors, data corresponding to a stress level of a user; determining a first time if the data from the one or more sensors can be greater than a corresponding data-threshold value, and commencing a treatment respondent to the first determination, wherein the treatment can include displaying a first visual program to the user and continuing to receive, from the one or more sensors, data corresponding to the stress level of the user; and terminating the treatment when the data from the one or more sensors can be lower than the corresponding data-threshold value.

The information about the stress level can include data can be selected from a group consisting of a heart rate, a temperature, a skin conductance, and a muscle activation. During operation the application can further determine information about a fatigue level of a human subject based on the received data. The application can be configured to determine information about the fatigue level of the human subject through operations can include: receiving, from one or more sensors, data corresponding to a fatigue level of a user; determining a first time if the data from the one or more sensors can be greater than a corresponding data-threshold value, and commencing a treatment respondent to the first determination, wherein the treatment can include displaying a first visual program to the user and continuing to receive, from the one or more sensors, data corresponding to the stress level of the user; and terminating the treatment when the data from the one or more sensors can be lower than the corresponding data-threshold value.

In a second aspect, disclosed herein is a device, including a casing defining an opening for receiving and encasing a mobile device; a temperature sensor housed by the casing; an infrared sensor housed by the casing; at least one conductance sensor housed by the casing; at least one electromyography sensor; a transceiver housed by the casing; and a control unit housed by the casing, the control unit being in communication with the temperature sensor, the infrared sensor, the at least one conductance sensor, the at least one electromyography sensor, and the transceiver, the control unit being programmed to receive data from the sensors and transmit, via the transceiver, the data to a mobile device.

In some embodiments, the device further can include a battery housed by the casing. The device further can include at least one communication port. The at least one electromyography sensor can include detachably secured to the device. The at least one electromyography sensor can include in wireless communication with the device when detached from the device.

The system can include multiple different types of sensor integrated into the cover of a user mobile device to acquire data in real time. Among other advantages, the combination of the system with a ubiquitous user mobile device can simplify the collection of multiple data parameters simultaneously and allow easy integration of the system into the daily routine of a user. Collected data is stored and the user can review their historical stress levels to help inform decisions about their relaxation habits. The system can also aid a user in determining an optimal range for various stress indicators, e.g., heart rate, or muscle activation, to achieve a state of optimum alertness without causing consequential negative health effects.

Other advantages will be apparent from the description, the drawings, and the claims.

### BRIEF DESCRIPTION OF THE DRAWINGS

FIG. 1 is a perspective view of the sensor surface of the system integrated in an example mobile device covering.

FIGS. 2A and 2B are edge views of a side and top of the system showing the orientation of components.

FIG. 3A is a view of the electromyography sensor in a compacted state.

FIG. 3B is a view of the electromyography sensor in an opened state, prior to affixing on a muscle group of the user.

FIG. 4 is a system diagram showing the communication paths of the system components.

FIG. 5 is a flowchart diagram of a first example controller threshold comparison process for determining to initiate user treatment.

FIG. 6 is a flowchart diagram of a second example controller threshold comparison process for determining length of user treatment.

FIG. 7 is a flowchart diagram for determining and treating a stress intensity level in a user.

FIG. 8 is a flowchart diagram of a controller threshold comparison process for determining and treating a fatigue level in a user.

In the figures, like symbols indicate like elements.

### DETAILED DESCRIPTION

This disclosure describes a stress-monitoring and treatment system designed to determine, monitor, and treat the stress level of an individual. The system uses sensors to measure parameters such as heart rate, skin conductivity, temperature, and muscle activation to determine the stress level of a user.

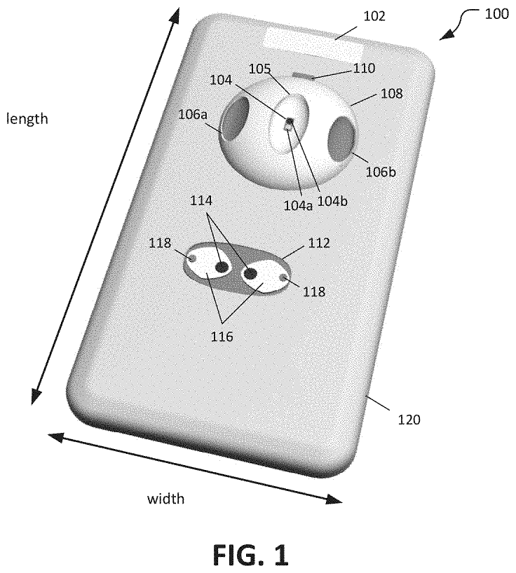

The stress-monitoring and treatment system includes an accessory for a mobile device of the user that includes a sensor module to detect, using one or more sensors of the sensor module, an elevated stress level of the user and a visual module to treat, responsive to the detection, the user with a visual program to attempt to reduce the stress level of the user. FIG. 1 is a perspective view of the sensor module 100 of the stress-monitoring and treatment system. The casing 120 is composed of a flexible or rigid material capable of protecting an encased mobile device from shock, impact, or damage such as silicone, rubber, or plastic. The curved edges of the casing 120 extend perpendicularly away from the sensor-containing surface of module 100 and provide an opening for receiving a mobile device (e.g., personal digital assistant, cellular telephone, smart-phone, tablet, or other similar computing device). When a mobile device is disposed within the opening, the casing 120 encases the mobile device and provides a detachable housing such that the interaction surface (e.g., screen, keypad, display, or touchscreen) of the mobile device remains accessible to the user.

The dimensions of the casing 120 are rectangular in shape with the length being greater than the width, and the width greater than the depth (length and width shown in FIG. 1). In some implementations, the length can be between 5 cm and 10 cm (e.g., between 7 cm and 10 cm, between 9 cm and 10 cm, or between 5 cm and 7 cm), the width can be between 3 cm and 7 cm (between 5 cm and 7 cm, or between 3 cm and 5 cm), and the depth can be between 0.5 cm and 1.5 cm (e.g., between 1.0 cm and 1.5 cm, or between 0.5 cm and 1 cm). The dimensions of the casing 120 can be constructed to match the dimensions of common mobile devices (e.g., iPhones) without changing the operation of the sensor module 100 of the system.

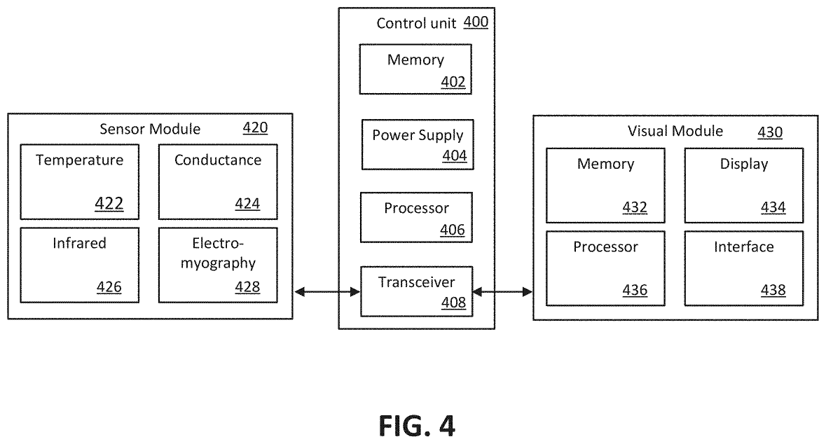

Sensor module 100 includes a number of sensors integrated into a common surface of the casing 120 to track biometric data of a user, e.g., a human subject. The sensor module 100 includes such as a temperature sensor 102, infrared (IR) sensor 104, one or more conductance sensors 106, and an electromyography (EMG) sensor array 112. The infrared sensor 104, and conductance sensors 106a, 106b integrate into a sensor housing 108. The sensor housing 108 further includes a communication port 110 for electronic communication with external peripherals connected to the system, such as a visual module. The interior volume of the sensor housing 108 contains electronic circuitry (e.g., a control unit) for receiving data from the respective sensors, signal processing, and communicating with the mobile device and attached peripherals. In some implementations, the sensor module 420 can communicate with and receive additional sensor data from additional sensors connecting to the communications port 110. The control unit is described in more detail with respect to FIG. 4.

FIG. 1 depicts an example orientation of the sensors included in sensor module 100, aligning along the longitudinal centerline of the casing 120. The positions and orientations of the respective sensors can be altered without changing the functionality of the module 100. The temperature sensor 102 is integrated with and disposed at a first end of the casing 120 and is a contact-type temperature sensor, such as a thermocouple (e.g., voltage-based) or thermistor (e.g., resistance-based). Temperature sensor 102 is in electronic communication with the control unit of the sensor housing 108 and the electric connection (e.g., the wire) is contained within the casing 120.

Referring now to the sensors housed in the sensor housing 108, a depression 105 is disposed in the center of the housing 108. Depression 105 recedes into the outer surface of the sensor housing 108 and forms a shape conforming to a finger pad of the user, e.g., an oval circumference and a hemispherical recession. Centrally disposed within the depression is an IR sensor 104.

IR sensor 104 is a photodiode sensor which includes an IR transmitter 104a and receiver 104b and determines the heart rate of a user. In operation, the user places the finger pad of a finger into the depression 105 and contacts IR sensor 104. When in contact with the finger pad, IR transmitter 104a emits light in the IR wavelength (e.g., between 780 nm and 1000 nm) which reflects off of the skin of the finger pad. Receiver 104b receives the reflected light thereby creating a signal. As blood pumps through vessels near the surface of the skin, receiver 104b measures changes in the reflected light signal. The control unit in sensor housing 108 determines a heart rate of the user based upon the signal changes.

Two conductance sensors 106a, 106b are disposed on opposing sides of depression 105, each contact area is composed of a conducting material such as aluminum, steel, or alloys thereof. When contacted by at least one finger pad, conductance sensors 106a,b measure user skin conductance which can be correlated with galvanic skin response (GSR) (e.g., conductance), a property of the human body that causes variation in the electrical characteristics of the skin wherein electrical conductance varies with the state of sweat glands in the skin. The sympathetic nervous system of the user controls the rate of sweat and balance of positive and negative ions in the fluid. If the sympathetic branch of the autonomic nervous system is highly aroused (e.g., high stress level), sweat gland activity increases which in turn increases skin conductivity. In operation, the user can contact the finger pad to one conductance sensor 106a or 106b independently, or two finger pads to both conductance sensors 106a and 106b concurrently.

The sensor module 100 further includes an electromyography (EMG) sensor array 112 including two surface EMG electrodes 114, each disposed within a respective adhesive pad 116. The EMG electrodes 114 detect the electric potential (e.g., voltage) generated by muscle cells when these cells are electrically or neurologically activated and EMG sensor array 112 measures the potential difference between more than one electrode 114 separated by a distance to generate EMG data supplied to the control unit. EMG measurements can quantify both muscle force and muscle activation (e.g., tension), indicators of the stress level of a user. FIG. 1 depicts the sensor array 112 as elliptical in shape, though other configurations can be considered, and detachably secures to a docking location on the casing 120.

The sensor array 112 houses a transceiver for communication with the sensor module 100 when the array 112 is detached from the module 100 and affixed to the skin of the user. For example, EMG sensor array 112 can house a wireless transceiver, such as a Bluetooth® transceiver, for wireless communication with the sensor module or the array 112 can be in wired communication with the module 100. The EMG sensor array 112 houses a power source (e.g., a battery) which supplies power to the components of the array 112 while detached from the sensor module 100 and a connection site to receive power from the sensor module 100 power supply when connected. In some implementations, the array 112 houses additional components to perform the function of signal acquisition and transmission including a voltage amplifier, a signal filtration unit, and/or an analog-to-digital (A/D) converter.

In some implementations, the sensor module 100 can receive sensor data from a wearable device of the user, including one or more integrated sensors. Examples of wearable devices can include smart watches, health tracking devices, or smart rings. The wearable device can communicate with the sensor module 420 through the mobile device or directly through the transceiver 408 of the control unit.

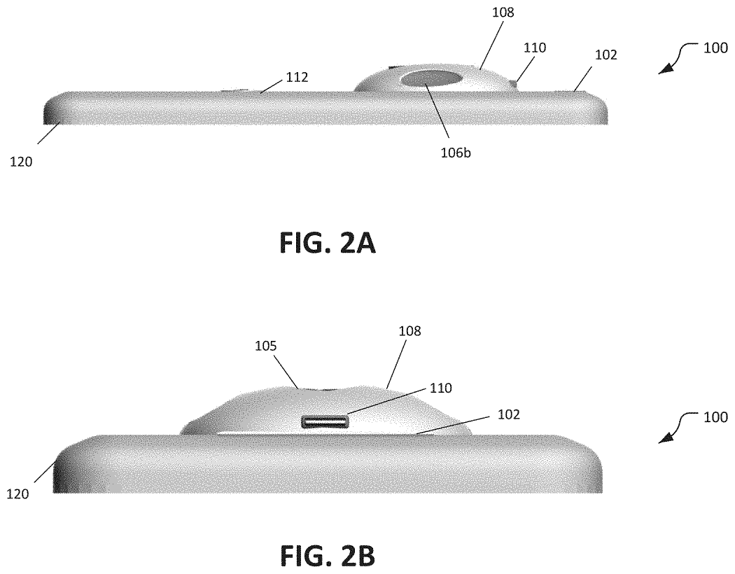

FIGS. 2A and 2B depict edge views of the sensor module 100 further illustrating the relative positioning of components detailed in FIG. 1. FIG. 2A is an edge view of the sensor module 100 with housing 108 oriented upward in the image. The casing 120 provides an opening opposite the housing 108 surface into which the user inserts the mobile device. From the right end of FIG. 2A, the temperature sensor 102 is disposed adjacent to the sensor housing 108, containing conductance sensor 106b and communications port 110. The EMG array is shown left-most in FIG. 2A.

FIG. 2B depicts an edge view of the module 100 from the end of the casing 120 at which the temperature sensor 102 is disposed. The sensor housing 108 protrudes from and centers on the longitudinal axis of sensor module 100 and communications port 110 orients toward the temperature sensor 102. The depression 105 housing the IR sensor 104 recedes into the central peak of the curvature of sensor housing 108.

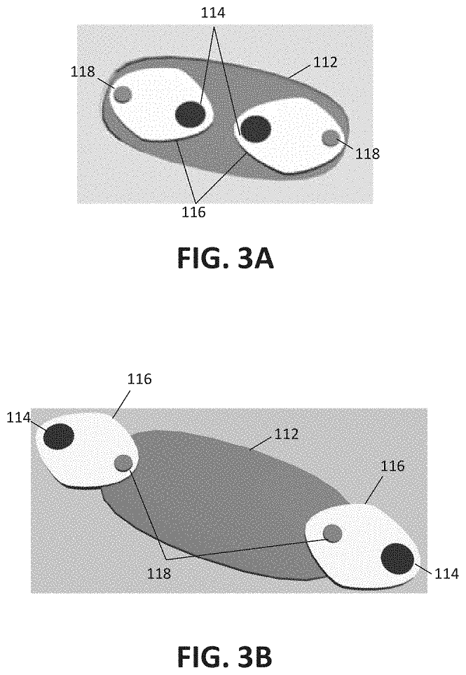

Turning now to FIGS. 3A and 3B, configuration details of the EMG sensor array 112 are shown. FIG. 3A depicts EMG sensor array 112 in a collapsed storage configuration, such as when the array 112 is secured to the docking location of casing 120. The array 112 includes two adhesive pads 116, each affixed to sensor array 112 via a fastener 118. Each pad 116 is elongate in shape and houses an EMG electrode 114 and corresponding electrical connections which pass through fastener 118. The fastener 118 connecting each pad 116 to the array 112 is disposed at an end nearest the outer edge of the array 112 and the EMG electrode 114 is disposed at the opposing end. The adhesive pad 116 rotates around the central axis of fastener 118.

Each pad 116 is composed of a flexible material, such as acrylic or high-density polyethylene. The EMG electrodes 114 are exposed on the outer surface of adhesive pads 116 and in some implementations, the surface of adhesive pads 116 that surrounds the electrodes 114 is treated with a skin adhesive (e.g., acrylate, methacrylates, epoxy diacrylates, or other vinyl resins) to adhere the adhesive pads 116 to the skin of the user.

In operation, the user detaches the EMG sensor array 112 from the casing 120 and rotates the pads approximately 180° to the position depicted in FIG. 3B. This configuration is not necessary but provides the largest distance between electrodes 114 while collecting data on muscle activation. The user adheres the array 112 to the skin above a muscle group by contacting the adhesive pads 116 to the skin. For example, the user adheres the array 112 above the trapezius muscle group of the neck and shoulders. The EMG electrodes 114 detect muscle activation and supply data to the array 112 which communicates the data to the control unit of the sensor module 100.

The sensors collect biometric data of the user and supply the data to the control unit housed within the sensor housing 108 for processing into signals. The control unit contains an algorithm for determining a stress level (e.g., a stress-determination algorithm) of a patient and whether a treatment will be performed. FIG. 4 is a block diagram of the control unit 400 depicting the communication channels between sensor module 420 and visual module 430 of the stress-monitoring and treatment system sensor module 100.

In some implementations, the control unit 400 can be a software application stored on the memory (e.g., an installed application, an app) of the mobile device encased in the casing 120. In such implementations, the control unit 400 utilizes the integrated memory, power supply, processor, and transceiver of the mobile device to receive data from the sensor module 420 and provide commands to the visual module 430. For example, the control unit 400 can store sensor data locally on the integrated memory of the user mobile device or use the transceiver of the mobile device to transfer the data to a networked location (e.g., a data server) for future presentation.

In implementations in which the control unit 400 is a computing device within the sensor housing 108, the control unit 400 includes computer memory 402, power supply 404, processor 406, and a transceiver 408. The memory 402 is generally one or more devices for storing data. The memory 402 can include long term stable data storage (e.g., on a hard disk), short term unstable (e.g., on Random Access Memory) or any other technologically appropriate configuration. In some implementations, the control unit 400 stores data received from the sensors within memory 402 for later presentation to the user via the visual module 430. The control unit 400 can use the stored data to establish a stress level through time (e.g., a historical stress level) of the user.

In general, the power supply 404 includes hardware used to receive electrical power from an outside source and supply it to components of the control unit 400. The power supply can include, for example, a battery pack. In some implementations, the power supply can further include ports to receive electrical power from a wall outlet adapter, an AC to DC converter, a DC to AC converter, a power conditioner, a capacitor bank, and/or one or more interfaces for providing power in the current type, voltage, etc., needed by other components of the control unit 400.

The processor 406 is generally a device for receiving input, performing logical operations on data, and providing output. The processor 406 can be a central processing unit, a microprocessor, general purpose logic circuity, application-specific integrated circuity, a combination of these, and/or other hardware for performing the functionality needed.

A transceiver 408 allows the control unit 400 to communicate with other components of the module 100, and user mobile device. For example, the control unit 400 communicates with sensor module 420, visual module 430, and user mobile device through the transceiver 408. The transceiver 408 can provide any technologically appropriate communication interface, including but not limited to multiple communication interfaces such as cellular, WiFi, Bluetooth®, and copper wired networks.

The sensor module 420 provides electronic communication between the sensors and control unit 400, of which module 100 is an example. The sensor module 420 receives data from the temperature sensor 422, the conductance sensor 424, the IR sensor 426, and the EMG sensor 428 and includes additional integrated components, such as microcontrollers, amplifiers, filters, and/or A/D converters, to process received data into signals which the module 420 supplies to the control unit 400. Sensor module 420 connects to and supplies signals via transceiver 408.

Visual module 430 includes computer memory 432, display 434, processor 436, and interface 438. The memory 432 and processor 436 are any such components as described herein. The memory 432 includes one or more visual programs for display to the user which, for example, can include still visual imagery (e.g., pictures), moving visual imagery (e.g., video), and/or auditory signals (e.g., music, or calming sounds).

Visual module 430 includes a graphical display 434 for displaying information to the user, such as sensor signals or visual programs. The graphical display can additionally be a display that the user can control by touching the screen with one or more fingers, e.g., a touchscreen. In some implementations, the visual module 430 is a head-mounted display for displaying virtual reality visual programs to the user.

In operation, the visual module 430 receives a command from the control unit 400 via interface 438 and processor 436 directs display 434 to display a visual program stored in memory 432 to the user. For example, the command that visual module 430 receives from control unit 400 can include a time duration. In response, the visual module 430 displays on the display 434 the visual program for the time duration received from the control unit 400.

In some implementations, the interface 438 allows a user to provide inputs or read outputs from the components of the control unit 400, sensor module 420, or visual module 430. The interface 438 can include a display, a visual program selection mechanism, and/or a sensor readout mechanism for selecting and displaying stress level information from one or more sensors. In some implementations, the mobile device of the user provides the interface 438.

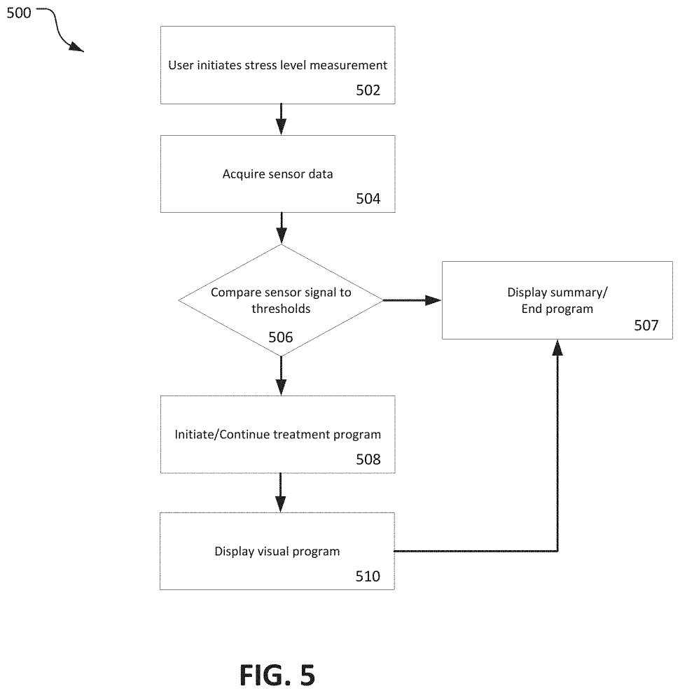

The control unit 400 holds in computer memory 402 an algorithm for determining and treating the stress level of the user, e.g., a stress-determination algorithm. FIG. 5 is a flowchart detailing an example process 500 the algorithm can follow to determine and treat the stress level.

The user initiates a stress level measurement (502) by contacting one or more sensors of the stress-monitoring and treatment system sensor module 100, such as temperature sensor 102, infrared (IR) sensor 104, conductance sensors 106, and/or EMG sensor array 112. In some implementations, the user initiates a stress level measurement by interacting with the visual module 430. For example, in some implementations, the user dons the head-mounted display which initiates the stress level measurement. The stress level measurement can occur for a time period during which sensor module 420 collects data from one or more sensors to establish a baseline for sensor values corresponding to the sensors contacted by the user.

The user contacts one or more sensors and the sensor module 420 receives sensor values (e.g., data) from the one or more sensors (504). For example, the user contacts the temperature sensor 102 which determines a temperature value and provides the value to the sensor module 420. In some implementations, the user wearable device can provide sensor values to the sensor module 420. The sensor module 420 processes and packages provided sensor values into a signal and supplies the signal to the control unit 400.

The control unit 400 holds in memory 402 corresponding threshold values for each sensor value received in signals from the sensor module 420. For example, the control unit 400 holds in memory 402 a temperature-threshold value, a heartbeat-threshold value, a skin conductance-threshold value, and a muscle activation-threshold value. The control unit 400 receives the signal from the sensor module 420, unpacks the signal into one or more sensor values, and compares the included sensor values to the corresponding threshold value (506), e.g., the temperature value to the temperature-threshold value. In some implementations, the threshold value is a baseline value (e.g., an average value) that the control unit 400 has determined from previously received values from the sensor module 420.

In some implementations, the user contacts one or more sensors over a period of time (e.g., minutes or hours) and the sensor module 420 receives a series of sensor values (e.g., a value every second, minute, or more) from the one or more sensors (504) (e.g., a series of temperature values). The sensor module 420 processes and packages the provided series of sensor values into a signal and supplies the signal to the control unit 400.

In such implementations, the control unit 400 determines a rate-of-change value from the series of sensor values. For example, the control unit 400 determines a temperature-rate-of-change value (e.g., 0.2° C. per minute, or 0.4° C. per minute) based upon a change between consecutive values of the series of temperature values. The control unit 400 holds in memory 402 corresponding rate-of-change threshold values for each sensor rate-of-change value received in signals from the sensor module 420. The control unit 400 holds in memory 402 a temperature-rate-of-change-threshold value, a heartbeat-rate-of-change-threshold value (e.g., 10 beats per minute per minute or more), a skin conductance-rate-of-change-threshold value (e.g., 0.2 μS per minute or more), and a muscle activation-rate-of-change-threshold value (e.g., 0.5 mV per minute or more). The control unit 400 receives the signal from the sensor module 420, unpacks the signal into one or more sensor values, determines a rate-of-change value and compares the included sensor values to the corresponding rate-of-change-threshold value, e.g., the temperature-rate-of-change value to the temperature-rate-of-change-threshold value.

If the control unit 400 determines that the received sensor value, or rate-of-change value, is not greater than the corresponding threshold value (e.g., a maximum threshold value), the control unit 400 does not execute a stress treatment program and displays on the display 434 of the visual module 430 a program end notification (507). The notification includes text indicating that the treatment program was not performed and can further include one or more sensor values, a computed stress level based upon at least the one or more sensor values, and/or a program end notification. In some implementations, the control unit 400 displays the notification on the mobile device of the user. In some implementations, the control unit 400 displays the notification on the wearable device of the user.

If the control unit 400 determines that one or more of the received sensor values, or rate-of-change values, is greater than the corresponding threshold value, the control unit 400 executes a stress treatment program (508) which includes supplying a command to the visual module 430 to display a visual program.

Processor 436 receives the command and instructs display 434 to display a visual program (510) received from memory 432. In some implementations, the command transmitted by the control unit 400 includes a time duration for the stress treatment program. In such implementations, the processor 436 receives the command including the time duration and instructs display 434 to display the visual program for the time duration. The control unit 400 then terminates the stress treatment program and displays the notification to the user (507).

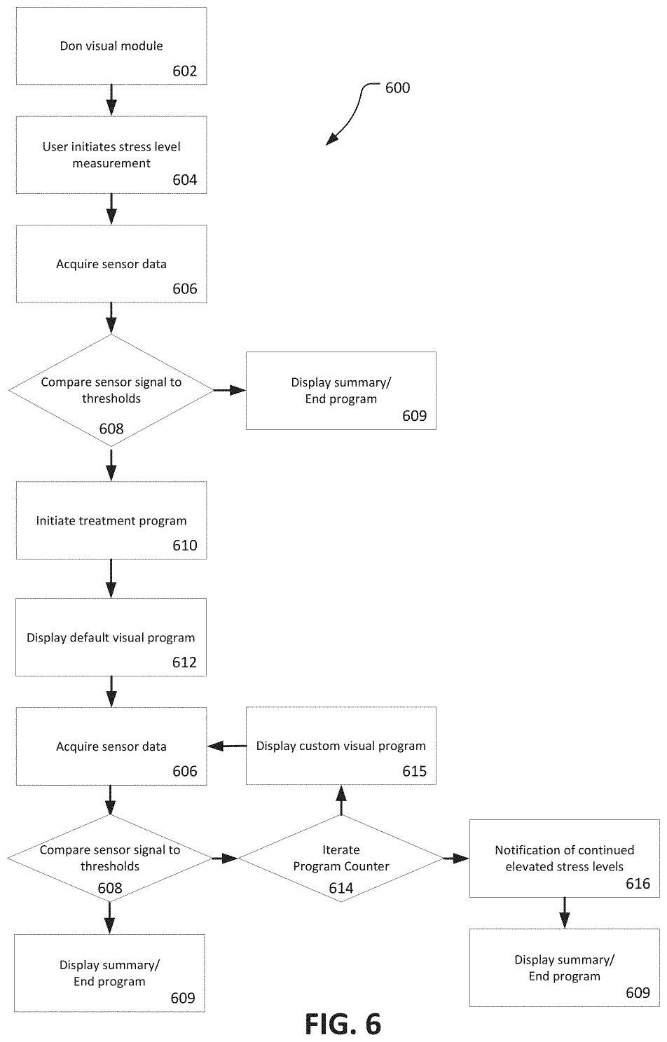

Treatment of the stress level of a user to elicit a relaxation response can include more than one visual program if the first does not reduce the stress level sufficiently. FIG. 6 is a flowchart detailing a second example process 600 the algorithm can follow to determine and treat the stress level of the user in which the control unit 400 includes a set number of visual programs to display prior to termination of the treatment program and initiates the stress level determination through the visual module 430.

The user dons the visual module 430 orienting the display 434 in front of the eyes. The visual module 430 sends a notification to the control unit 400 that the visual module 430 is in use by the user.

Donning the visual module 430 initiates a stress level measurement (604). In some implementations, the user initiates the stress level measurement by interacting with the interface 438 of the visual module. During the measurement, the user can contact one or more sensors (606) to collect additional data corresponding to the stress level of the user, as in step (502) of FIG. 5.

The control unit 400 receives the signals from the sensor module 420, unpacks the signal into one or more sensor values, and compares the included sensor values to the corresponding threshold value (608), e.g., the temperature value to the temperature-threshold value, as in step (506) of FIG. 5.

If the control unit 400 determines that the received sensor value is not greater than the corresponding threshold value, described above, the control unit 400 does not execute a stress treatment program and displays on the display 434 of the visual module 430 a program end notification (609) indicating that the treatment program was not performed.

If the control unit 400 determines that one or more of the received sensor values is greater than the corresponding threshold value, the control unit 400 executes a stress treatment program (610) to evoke a relaxation response in the user. The initial stress treatment program includes a default visual program displayed (612) to the user, as described above. In some implementations, the control unit 400 keeps in memory 402 a program counter (e.g., N) corresponding to the number of programs that have been displayed to the user. For example, during the initial stress treatment program, the program counter iterates to a value of one (e.g., N=1).

While the visual module 430 displays the initial visual program, the control unit 400 contemporaneously receives signals (606) from the sensor module 420 including sensor values from one or more sensors in contact with the user. After completion of the visual program, the control unit 400 compares (608) signal values received during the initial visual treatment program to corresponding threshold values. If the signal values are less than the threshold values, the control unit 400 terminates the stress treatment program and displays the notification to the user (609).

If the signal values are greater than corresponding threshold values, the control unit 400 commands the visual module 430 to execute an additional visual program and the program counter iterates (e.g., N=2) (614). Additional visual programs can be custom programs (615) designed specifically to induce a relaxation response in the user. This can include, for example, user-selected visual and/or audio information or a program designed by a health practitioner to elicit a relaxation response.

While the visual module 430 displays the custom visual program, the sensor module 420 contemporaneously acquires sensor data (606) from the one or more sensor the user contacts. Following the completion of the additional programs, the control unit 400 compares (608) the sensor module 420 signal values to the corresponding threshold values and either terminates the stress treatment program and displays the notification to the user (609) or determines further programs to be displayed to the user. The counter is iterated (614) and additional visual programs are displayed (615).

Steps 606, 608, 614, and 615 constitute a loop through which the control unit will iterate until either the elicited relaxation response reduces the sensor values below the corresponding threshold levels or program counter reaches a maximum program threshold-value stored in memory 402. The maximum program threshold-value (e.g., N_max) is a user—or app—defined value which limits the number of visual programs displayed to a user by the visual module 430.

When the program counter is equal to the program threshold-value (e.g., N=N_max), the control unit 400 terminates the stress treatment program and commands the visual module 430 to display a final notification indicating the end of the stress management program. The final notification can include information regarding the continued elevated stress level (616) of the user at the termination of the stress management program, such as sensor data received from the sensor module 420 during the final visual program, or a prompt for display to the user, such as a prompt advising communication with a health professional. Additionally, the visual module can display the termination notification (609) to the user.

The processes of FIGS. 5 and 6 describe examples in which the stress treatment program seeks to reduce the stress level of the user. However, the system can include minimum sensor threshold values as well as maximum defining a region of sensor values between the maximum and minimum as a preferable stress level. In such implementations, the control unit 400 can determine that received sensor values from the sensor module 420 are less than the minimum threshold-values and command the visual module 430 to display to the user a visual program designed to increase stress levels (e.g., to elicit an arousal response).

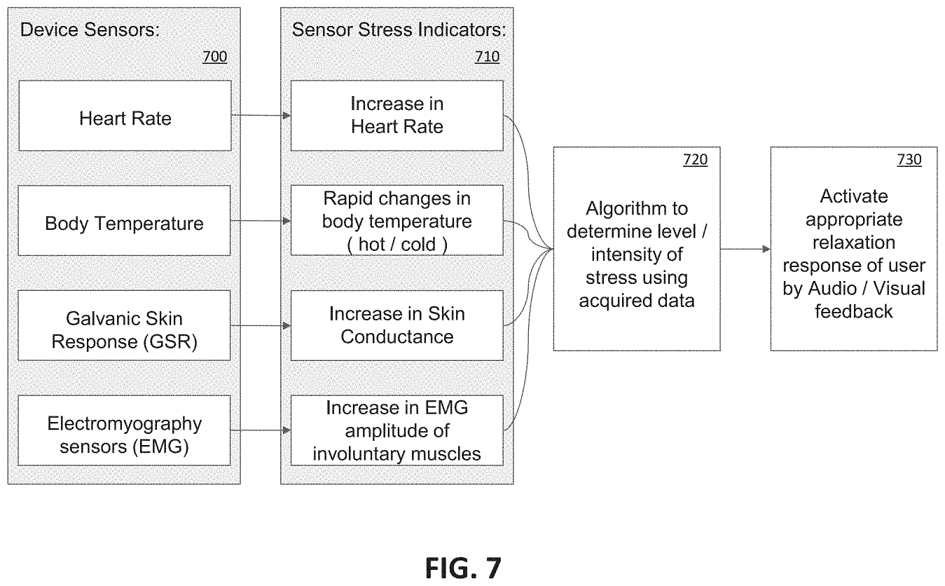

FIG. 7 is an exemplary flow diagram detailing a stress indicator determination process using the information collected from sensor module 420. The values are compared to the corresponding threshold values and stress level indicators are determined and fed into an algorithm determining the stress level of a user. These determinations then activate appropriate relaxation responses from the devices.

Table 700 shows the sensor values the control unit 400 receives from the sensor module 420: a heart rate value provided from the IR sensor 104, a temperature value provided from the temperature sensor 102, and GSR value provided from the conductance sensors 106a, 106b, and a muscle activation value from the EMG sensor array 112. Each sensor value is received by the control unit 400 and compared to threshold values, as described above.

Table 710 shows example stress level indicators that the control unit 400 determines from received sensor values when compared to corresponding threshold values. Each stress level indicator can indicate a state of elevated stress (e.g., arousal) in the user and the number of indicators having been met can indicate the level of elevated stress. For example, control unit 400 can receive from the IR sensor 104 values indicating an increased heart rate of a user (e.g., 100 beats per minute, 110 beats per minute, 120 beats per minute or more), values from the temperature sensor 102 indicating increased, or rapid changes in, surface temperature of the user (e.g., 0.5° F. per minute or more), values from conductance sensors 106a, 106b indicating an increase in skin conductance (e.g., 20 μS, 25 μS, 30 μS or more), or increased EMG value amplitude (e.g., 8 mV, 10 mV, 12 mV or more).

From the stress level indicators in table 710, the control unit 400 algorithm determines a stress intensity level (720) from the number of indicators with exceeded threshold values. The control unit 400 determines a stress intensity level based upon at least the stress level indicators of table 720. In some implementations, the control unit 400 classifies the stress intensity level as low, moderate, or high, though other classifications are possible. For example, a single sensor value greater than a corresponding threshold value may indicate a low stress intensity level whereas more than two sensor values greater than a corresponding threshold value may indicate a high stress intensity level.

Determining the stress intensity level allows the control unit 410 to command the visual module 430 to display a visual program corresponding with the determined level (730). The visual module 430 receives the command from the control unit 400 and displays a visual program stored in memory 432 designed to treat the determined stress intensity level.

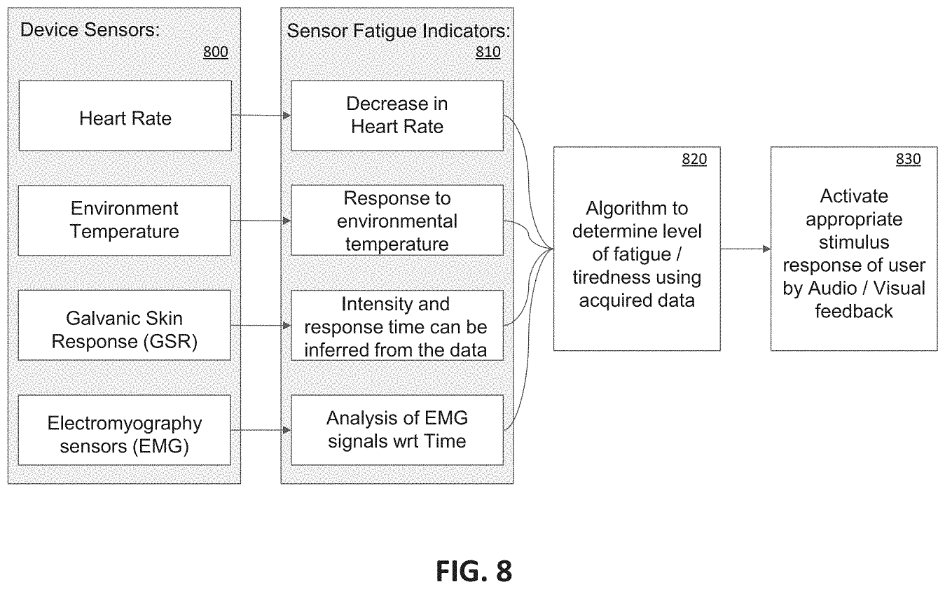

In some implementations, the sensor module 100 determines and treats a fatigue level (e.g., tiredness) of the user. FIG. 8 is an exemplary information flow diagram of collected sensor values, comparison to fatigue level indicators, and determination and treatment of user fatigue levels.

Table 800 shows the sensor values the control unit 400 receives from the sensor module 420, similar to table 700, including a heart rate value, a temperature value, a GSR value, and a muscle activation value. Table 810 depicts the corresponding fatigue level indicators and corresponding threshold values the control unit 400 compares to the received sensor values to make a fatigue level determination. For example, a received heart rate value indicating a decreased heart rate (e.g., 70 beats per minute, 60 beats per minute, 50 beats per minute), a temperature value indicating a decreased surface temperature (e.g., 98° F. or less, 97° F. or less), a conductance value indicating increased amplitude (e.g., 20 μS or more, 25 μS or more, 30 μS or more) or response times (e.g., periodic fluctuations in conductance values), or decreased EMG value amplitude (e.g., 10 mV or less, 7 mV or less, 6 mV or less, 5 mV or less).

The control unit 400 includes an algorithm for determining a fatigue level of a user, and from the number of fatigue indicators with exceeded threshold values determines a fatigue level (820). For example, a single fatigue indication may indicate a low fatigue level whereas more than two fatigue indications may indicate a high fatigue level.

Determining the fatigue level allows the control unit 410 to command the visual module 430 to display a visual program corresponding with the determined fatigue level (830). The visual module 430 receives the command from the control unit 400 and displays a visual program stored in memory 432 designed to treat the determined fatigue level, as described above.

Other embodiments are in the following claims.

What is claimed is:

1. A system, comprising:
a mobile device;
a sensor module attached to the mobile device, the sensor module comprising a temperature sensor, an infrared sensor, at least one conductance sensor, and at least one electromyography sensor; and
an application installed on the mobile device, wherein during operation the application causes the mobile device to receive data from one or more of the temperature sensor, the infrared sensor, the at least one conductance sensor, and the at least one electromyography sensor, and to determine information about a stress level of a human subject based on the received data.

2. The system of claim 1, the sensor module further comprising a casing defining an opening for receiving and encasing the mobile device.

3. The system of claim 2, the sensor module further comprising a transceiver housed by the casing.

4. The system of claim 3, the electromyography sensor being detachably secured to the sensor module.

5. The system of claim 4, wherein the electromyography sensor is in wireless communication with the sensor module when detached from the mobile device.

6. The system of claim 4, wherein the electromyography sensor is in wired communication with the sensor module.

7. The system of claim 1, further comprising a visual module for displaying to the human subject a treatment program, comprising at least one processor, and a display, wherein the visual module receives a command from the device to play the treatment program on the display.

8. The system of claim 7, the treatment program comprising a visual program, or an audio program.

9. The system of claim 1, wherein the application is configured to determine information about the stress level of the human subject through operations comprising:
receiving, from one or more sensors, data corresponding to a stress level of a user;
determining a first time if the data from the one or more sensors is greater than a corresponding data-threshold value, and commencing a treatment respondent to the first determination, wherein the treatment comprises displaying a first visual program to the user and continuing to receive, from the one or more sensors, data corresponding to the stress level of the user; and
terminating the treatment when the data from the one or more sensors is lower than the corresponding data-threshold value.

10. The system of claim 9, wherein the information about the stress level comprises data selected from a group consisting of a heart rate, a temperature, a skin conductance, and a muscle activation.

11. The system of claim 1, wherein during operation the application further determines information about a fatigue level of a human subject based on the received data.

12. The system of claim 11, wherein the application is configured to determine information about the fatigue level of the human subject through operations comprising:
receiving, from one or more sensors, data corresponding to a fatigue level of a user;
determining a first time if the data from the one or more sensors is greater than a corresponding data-threshold value, and commencing a treatment respondent to the first determination, wherein the treatment comprises displaying a first visual program to the user and continuing to receive, from the one or more sensors, data corresponding to the stress level of the user; and
terminating the treatment when the data from the one or more sensors is lower than the corresponding data-threshold value.

13. A device, comprising:
a casing defining an opening for receiving and encasing a mobile device;
a temperature sensor housed by the casing;
an infrared sensor housed by the casing;
at least one conductance sensor housed by the casing;
at least one electromyography sensor;
a transceiver housed by the casing; and
a control unit housed by the casing, the control unit being in communication with the temperature sensor, the infrared sensor, the at least one conductance sensor, the at least one electromyography sensor, and the transceiver, the control unit being programmed to receive data from the sensors and transmit, via the transceiver, the data to a mobile device.

14. The device of claim 13, further comprising a battery housed by the casing.

15. The device of claim 14, further comprising at least one communication port.

16. The device of claim 13, wherein the at least one electromyography sensor is detachably secured to the device.

17. The device of claim 16, wherein the at least one electromyography sensor is in wireless communication with the device when detached from the device.
